(19) United States
(12) Patent Application Publication
(10) Pub. No.: US 2007/0083232 A1
(43) Pub. Date: Apr. 12, 2007

(54) Vascular closure device

(75) Inventor: Benjamin Lee, Chevy Chase, MD (US)

Correspondence Address: FOLEY AND LARDNER LLP;SUITE 500, 3000 K STREET NW, WASHINGTON, DC, 20007, US

(73) Assignee: Innovasive, Inc.

(21) Appl. No.: 11/437,834

(22) Filed: May 22, 2006

Related U.S. Application Data
(63) Continuation-in-part of application No. 11/245,437, filed on Oct. 7, 2005.

(51) Int. Cl. A61B 17/08

(52) U.S. Cl. 606/213

(57) ABSTRACT

The present invention provides a device for closing an opening to a body cavity and methods of closing an opening to a body cavity. The device and methods can be used to easily and effectively close a vascular puncture site resulting from a surgical procedure, an atrial or ventricular septal defect, a malfunctioning heart valve, or the left atrium appendage.

This application is a continuation-in-part of application Ser. No. 11/245,437 filed on Oct. 7, 2005. The present invention relates to methods and devices used for closing openings in a body lumen, such as puncture sites in blood vessels, septal defects in heart chambers, heart valves that do not seal, and the mouth of the left atrial appendage.

### BACKGROUND OF THE INVENTION

A growing number of therapeutic and diagnostic medical procedures involve the percutaneous introduction of instrumentation into a vein or artery. For example, in the treatment of vascular disease, such as atherosclerosis, it is a common practice to insert an instrument, such as a balloon, into an artery to carry out the procedure within the artery. Although a physician may elect to use a balloon to stretch out a vessel, he may alternatively use a laser to burn through any plaque present and open up the artery. Also, the physician may inject clot dissolving chemicals directly into the blocked artery or may remove the clot directly with special instruments. In addition, physicians often insert stents into a vessel to keep it open. In any case, a vessel is pierced in some way to allow access to the vessel interior.

The closing and subsequent healing of the resultant vascular puncture is critical to the successful completion of the procedure. Traditionally, the application of external pressure to the skin entry site, followed by patient immobility, has been employed to stem bleeding from the wound until clotting and tissue rebuilding have sealed the perforation. With externally-applied manual pressure, not only is patient comfort impaired, but practitioners are not being utilized efficiently. In the case of punctures into femoral or superficial femoral arteries, the pressure may have to be applied for extended periods of time for hemostasis to occur. Additionally, a risk of hematoma exists, since bleeding from the vessel may continue until sufficient clotting effects hemostasis. Not only is direct pressure inefficient from both a medical and personnel perspective, the procedure may result in substantial reduction, if not complete arrest, of the flow of blood through the vessel. Since thrombosis is one of the major calamities that can occur in the post-operative period, any reduction in blood flow is undesirable. Also, external pressure application devices may be unsuitable for patients with substantial amounts of subcutaneous adipose tissue, since the skin surface may be a considerable distance from the vascular puncture site, thereby rendering skin compression inaccurate and thus less effective.

Consequently, devices have been developed for promoting hemostasis directly at the site of the vascular perforation. For example, there are devices that deploy intraluminal plugs within the vessel to close the puncture site. Another approach is to deliver tissue adhesive or clotting agent to the perforation site. This method may entail some risk of disadvantageously introducing some of the adhesive or clotting agent into the bloodstream. Still another approach is the application of pressure directly to the perforation site. Yet another approach is where a cylindrical plug is inserted along the shaft of a catheter segment extending from the skin surface to the blood vessel. The catheter is then removed so that the plug can expand as fluid is drawn into the plug from the vessel and the surrounding tissue. Unless pressure is applied, however, bleeding may occur around the plug into the subcutaneous tissue. A variety of plug delivery devices are exemplified by threaded plug pushers and multilegged channels, which install a plug that may be resorbable.

Many of the above-noted devices rely, to varying degrees, on tactile sensation alone to indicate to the surgeon the proper placement of the puncture closing instrumentation, and they may also require upstream clamping of the blood vessel to reduce intraluminal pressure to approximately atmospheric pressure at the puncture site. In fact, many of these techniques require a great deal of experience and manual dexterity to use successfully. Thus, even experienced surgeons can have difficulty in using these techniques and devices.

Another type of percutaneous vascular hemostasis device comprises a mechanism for delivering a suture percutaneously to a vascular suturing site, and then tying the suture in situ. While such devices, if properly employed, are capable of very effectively stemming blood flow, they may require a relatively high degree of dexterity to be operated properly. Indeed, the vessel opening is often accessible through only small catheters making sutures even more difficult to tie. Furthermore, the devices tend to be somewhat complex and expensive to manufacture, and thus are not practically employed as single use, disposable products. Consequently, sterilization is required between uses to reduce the risk of infection, thereby increasing their cost and inconvenience.

Accordingly, there has been a long-felt need for an effective percutaneous vascular hemostasis device that is relatively simple and inexpensive to manufacture and easy to use, that does not require prohibitively precise dexterity, that is adapted for use as a disposable device, and that does not require the introduction of a foreign substance, such as a plug, a tissue adhesive, or a clotting agent, into the bloodstream. An ideal device would exploit modern advances, but would also apply some external pressure on the puncture site itself, which would also serve to seal the puncture.

In addition to an effective percutaneous vascular hemostasis device, there is also a need for a device that can be used to close intracardiac defects (i.e., patent foramen ovale (PFO), atrial, and ventricular septal defects), heart valve repair (i.e., for mitral and tricuspid insufficiency), and obliteration of the left atrial appendage (LAA) to lessen the risk of stroke. While these applications all differ to some degree, all are similar in that they involve the closure of a body opening.

Current devices for performing these functions suffer many of the same limitations as existing vascular closure devices. For example, current devices for PFO closure are the CardioSEAL® Occluder and the StarFlex® Occluder. These devices, like many others on the market, are essentially patches that must be positioned at the PFO and occlude the hole. In an example of repairing the mitral valve, one device that has been developed is the MitraClip™ in which a catheter is implanted into the left atrium of the heart and a clip is used to grasp and hold the center of the valve leaflets closed while allowing blood to flow on either side of the clamped center. In an example for obliterating the left atrial appendage (LAA), current devices essentially plug the left atrial appendage with some foreign material, such as a coiled wire. Other examples of devices and methods of closing the LAA are disclosed in U.S. Pat. No. 6,152,144. All of these devices can potentially come loose, which would likely result in death, and there is a significant risk of thrombosis due to the implantation of foreign material.

Thus, devices currently in use for closing body openings, such as blood vessels, ventricular and atrial defects, heart valves, and left atrium appendages, rely largely on the tactile sensation and skill of the surgeon. Accordingly, even experienced surgeons may have difficulty using the devices increasing the risk of surgical complications. In addition, existing devices are relatively expensive. Thus, a need exists for devices that can close intracardiac defects, effect heart valve repair, and obliterate the LAA quickly, effectively, easily, and cheaply.

### SUMMARY OF THE INVENTION

The present invention relates a closure device for effectively sealing a blood vessel or other body opening, and the structure and method of its introduction, application, and extraction. Embodiments of the present invention require little manual dexterity, are easy to use, and effectively seal a perforation by using three complementary methods: using grasping tines to appose the edges of the perforation together, folding the edges tightly together rather than simply pulling them together, and plugging the perforation site with a collar. Because embodiments of the present invention use three mechanisms to effectively seal a perforation, they provide an excellent seal and enable faster healing. Also, because they are particularly inexpensive to manufacture, they are especially well-suited for one-time use, making post-surgical sterilization unnecessary, thus cutting costs and increasing convenience.

It is an object of the present invention to fulfill one or more of the needs referred to above. In accordance with the principles of the present invention, this objective is obtained by providing a device and method for closing and sealing a puncture in a luminal wall. Embodiments of the present invention, in one aspect, provide a reliable and easily used device for promoting and achieving percutaneous vascular hemostasis at a perforation or puncture site in a subcutaneous bodily lumen, especially a blood vessel or a heart chamber, using a combination of sealing mechanisms to promote hemostasis in the most effective manner. In another aspect, the present invention relates to the method of using this hemostasis device to promote hemostasis at such a site.

In one embodiment, the present invention provides for a closure device comprising at least two tines including a distal end and a proximal end, wherein the tines are joined at the proximal end, and wherein each tine has at least one barb adapted for catching tissue; a collar with an opening receiving the proximal end of the resilient tines; wherein the tines expand radially outward to an open position when unrestricted, and wherein the collar can be moved from the proximal end of the tines towards the distal end of the tines to radially contract the tines to a closed position.

In another embodiment, the present invention provides for a device for closing a vascular opening or other body cavity opening comprising: at least two tines including a distal end and a proximal end, wherein the tines are joined at the proximal end, and wherein the tines each have at least one barb adapted for catching tissue, and wherein the tines are deflected outward from the longitudinal axis of the tines; and a collar with an opening receiving the proximal end of the tines; wherein the collar can be moved from the proximal end of the tines towards the distal end of the two resilient tines to radially contract the tines, thereby decreasing the deflection.

In an additional embodiment, the present invention provides for a vascular closure device comprising a tube with an open distal end; a collar disposed on the distal end of the tube and including an opening for receiving at least two tines, wherein the collar is sized to be pushed by the tube when the tube is moved distally; at least two tines, each of the tines inwardly collapsible to be received in an arterial sheath and with at least one barb adapted for grasping vascular tissue, wherein the tines are moveably connected to the opening of the collar whereby, when unrestricted, the tines expand radially outward to an open position, and when the tube is moved distally, the collar is moved distally thereby moving the tines into the opening of the collar to radially contract the tines to a closed position.

In one embodiment, the present invention provides a method of closing an opening to a body cavity comprising advancing at least two resilient tines through a body opening into a body lumen, wherein the tines each have at least one barb adapted for catching on tissue and wherein the tines have an open position wherein the tines are radially expanded and a closed position wherein tines are radially contracted; retracting the tines in the open position against the interior tissue of the lumen, wherein the barb catches on the tissue adjacent to the body opening; radially contracting the tines to the closed position wherein contracting pulls the edges of the tissue adjacent opening together to close the body opening; and securing the tines in the closed position.

In another embodiment, the present invention provides a method for promoting hemostasis at a vascular opening comprising providing percutaneous access to the tissue opening through an arterial sheath with an open distal end disposed within the vascular lumen and an open proximal end; providing at least two tines, wherein each tine is inwardly collapsible to be received in an arterial sheath and each tine has at least one barb for grasping vascular tissue, wherein the tines have an open position wherein the tines are radially expanded, and a closed position wherein the tines are radially contracted into the sheath; advancing the tines through the sheath and into the vascular lumen so that the tines expand outwardly in the vascular lumen; retracting the tines so that the tines are pulled against the interior surface of the vascular lumen, wherein the tines catch on the tissue forming the interior surface of the vascular lumen; and advancing a collar to the exterior surface of the vascular opening, wherein the collar causes the tines to radially contract in a manner to pull the edges of the vascular tissue together.

It is to be understood that both the foregoing general description and the following detailed description are exemplary and explanatory only, and are not restrictive of the invention as claimed. These and other objects of the present invention will be apparent to one of ordinary skill in the art.

### BRIEF DESCRIPTION OF THE DRAWINGS

FIG. 1 shows a sectional view of a puncture site in a blood vessel with the vascular closure device, according to an embodiment of the present invention.

FIG. 2 shows a sectional view of a puncture site in a blood vessel with the vascular closure device, according to an embodiment of the present invention.

FIG. 3 shows a sectional view of a puncture site in a blood vessel with the vascular closure device, according to an embodiment of the present invention.

FIG. 4 shows a sectional view of a puncture site in a blood vessel that has been closed with the vascular closure device, according to an embodiment of the present invention.

FIG. 5 shows an internal sectional view of a puncture site in a blood vessel.

FIG. 6 shows an internal sectional view of a puncture site in a blood vessel in which the vascular closure device has been inserted, according to an embodiment of the present invention.

FIG. 7 shows an internal sectional view of a puncture site in a blood vessel that has been closed with the vascular closure device, according to an embodiment of the present invention.

FIG. 8 shows a sectional view of a puncture site in a blood vessel with the vascular closure device, according to an embodiment of the present invention.

FIG. 9 shows a sectional view of a puncture site in a blood vessel with the vascular closure device, according to an embodiment of the present invention.

FIG. 10 shows a sectional view of a puncture site in a blood vessel with the vascular closure device, according to an embodiment of the present invention.

FIG. 11 shows a sectional view of a puncture site in a blood vessel with the vascular closure device, according to an embodiment of the present invention.

FIGS. 12(a)-12(c) show a sectional view of an atrial septal defect site in a heart with the vascular closure device and the method for closing the defect, according to an embodiment of the present invention.

FIGS. 13(a)-13(c) show a sectional view of a ventricular septal defect site in a heart with the vascular closure device and the method for closing the defect, according to an embodiment of the present invention.

FIGS. 14(a)-14(e) show a sectional view of the method for repairing the mitral valve in a heart with the vascular closure device, according to an embodiment of the present invention.

FIGS. 15(a)-15(d) show a sectional view of the method for repairing the tricuspid valve in a heart with the vascular closure device, according to an embodiment of the present invention.

FIGS. 16(a)-16(d) show a sectional view of the method for closing the left atrium appendage in a heart with the vascular closure device, according to an embodiment of the present invention.

### DETAILED DESCRIPTION OF THE INVENTION

The present invention provides a reliable and easily-used vascular closure device for closing and effectively sealing an opening in a luminal wall, such as made during the course of a percutaneous surgical procedure, using three complementary sealing methods. The device comprises at least two resilient tines and a collar. The practitioner contracts the resilient tines from an open state to a closed state to grasp the interior edges of the opening together and folds and apposes the edges tightly together. The collar is then used to hold the tines in a closed position. In addition, the collar acts to plug the opening thereby acting as another means of sealing the opening. Because the present invention uses three mechanisms to effectively seal a perforation, it provides a better seal, enables faster healing, and better promotes and achieves percutaneous vascular hemostasis allowing earlier ambulation and patient discharge in the most effective manner. In addition, the use of the device requires little manual dexterity and can therefore be used quickly and easily by even inexperienced practitioners.

Embodiments of the present invention will be described below with reference to the drawings.

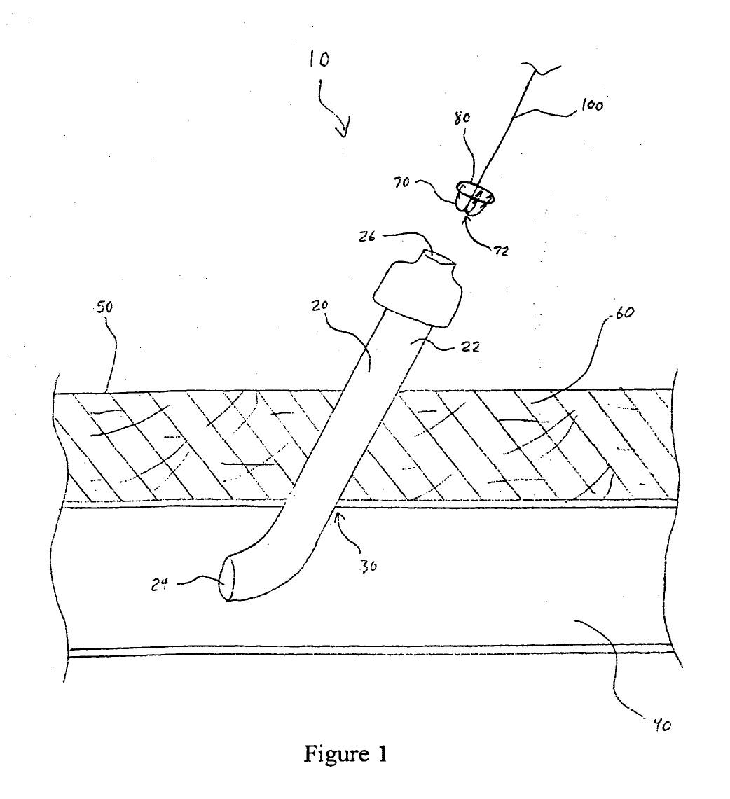

FIG. 1 shows a vascular closure device 10, in accordance with a first preferred embodiment of the present invention, that may include an arterial sheath, a surgical sheath, or a trocar 20 deployed at a perforation or puncture site 30 in a subcutaneous bodily lumen 40. For the purposes of the ensuing discussion, the lumen 40 will be referred to below as a blood vessel, although the adaptation of the present invention for use in procedures involving other organs will readily suggest itself to those skilled in the pertinent arts. For the purposes of the following discussion, the term blood vessel may include veins, arteries, and similar tissues.

The sheath 20 can be a conventional type, commonly used in surgical procedures, and, as shown in FIG. 1, it is positioned as it would be after the completion of such a surgical procedure. Specifically, the sheath 20 may include an elongate hollow tube or barrel 22 that is inserted through the skin 50 and subcutaneous tissue 60, with an open internal or distal end 24 that has been inserted into the vessel 40 through the puncture site 30. The barrel 22 has an open external or proximal end 26 that extends from a surgical entry site in the skin 50.

In a first preferred embodiment, the vascular closure device 10 comprises at least two tines 70 attached to a suture or wire 100, and a collar 80. More preferably, two pairs of tines 70 may be joined at their proximal ends to the wire 100. The device may have two, three, four, five, or more tines, whether positioned in pairs or not. For putting the present invention into practice, a metal alloy can be used, and one preferred embodiment would be made of a magnesium alloy. As mentioned in U.S. Pat. No. 6,287,332, and incorporated herein as a reference, lithium-magnesium alloys can be used, which have a lower fatigue durability during conventional treatment and in the body sphere. Lithium hydroxide and magnesium hydroxide are to be expected as decomposition products, but can both be considered non-toxic and biocompatible.

The tines 70 may be made to have a naturally open position in which they diverge radially outwardly, and their natural resilience causes them to return to this naturally open position if they are squeezed together (i.e. radially inward) and then released. This property can be achieved by using a shape memory alloy, as described in U.S. Pat. No. 5,002,563, which is hereby incorporated by reference. Shape memory alloys are metals that exhibit the properties of pseudo-elasticity and shape memory effect; they have been used in such machines as coffeepots, the space shuttle, and thermostats, and are in use in surgical devices such as bone plates and vascular stents. Other types of materials, such as polymers and metals, can also be used to make the tines 70. In fact, the tines 70 can be made of several materials, such as a rigid material to form the body of the tines 70 and a flexible material that can be used in appropriate places to give the tines 70 the desired shape.

The tines 70 can be joined at their proximal ends 72 using any suitable method. For example, the tines 70 may be welded together or joined using an adhesive. In some embodiments, the tines 70 can be molded, so the tines 70 are formed as a single entity joined at the proximal end 72 during manufacturing. In one embodiment, the joined tines 70 may be in turn joined to a suture or wire 100. In another embodiment, the tines 70 may be integral to the wire 100.

When a suture is used, the suture 100 may be a cord made of a bioresorbable material. Furthermore, the suture 100 may be made of vicryl, polydioxalone, polypropylene, nylon, silk, and steel. The tines 70 may be connected to the suture 100 by a knot or by an adhesive.

In some embodiments, the tines 70 are joined in such a manner that their connection is reversible, such as a bioresorbable material. Accordingly, the joint can be designed to break at a predetermined time, such as after positioning, or the joint can be designed to be easily broken by the practitioner at the completion of positioning the device.

Each of the tines 70 has an inwardly-turned barb or hook 75 at the distal end of the tine. Any suitable barb design can be employed, so long as the barb 75 is effective in attaching to or hooking tissue. For example, the barbs 75 can be designed like the barb on a fish hook. Other designs will be readily apparent to one of skill in the art. The function of the barbs will be explained below.

The device 10 also comprises a slidable locking collar 80. The distal ends of the tines may be received in the collar 80, which can be pushed distally over the tines 70 to lock the tines 70 into their closed position. As will be made clear from the explanation of the operation of the device 10 as set forth below, the tines 70 and the collar 80 advantageously can be made of a bioresorbable material. Bioresorbable materials are well-known in the art and can be readily selected by a skilled artisan.

A bioresorbable material is a resorbable material that is biocompatible; biocompatible material is compatible with a living system or living tissue, is non-toxic or non-injurious, and does not cause immunological reaction or rejection. A number of such materials will exhibit the requisite degree of resilience to provide the self-opening tine function described above, such as those described in U.S. Pat. No. 5,919,234 or U.S. Pat. No. 6,786,910, which are hereby incorporated as references. Any appropriate biodegradable, bioerodible, or bioresorbable materials can be used, so long as they have the desired characteristics, including biocompatibility, flexibility, and strength. Although the materials described in U.S. Pat. No. 5,919,234 are perforated, that is not a requirement of the present invention. As described in U.S. Pat. No. 6,786,910, bioresorbable refers to a structure or material that, over time, can be at least partially removed by biological action within the body of a subject. Bioresorbable material can include a bioactive compound, such as a pharmaceutical composition, a protein, a peptide, a nucleic acid molecule or a small molecule. Such bioactive compounds preferably have desirable activities associated with distraction procedures, such as growth factors of various types, bone morphogenic proteins, antibiotics or other compounds to improve or hasten the bone consolidation period or to decrease the time of distraction. These bioactive compounds can be leached from the bioresorbable materials over time or be released as the biodegradable materials are removed by biological action. The bioresorbable materials, if any, used with the present invention should be selected based on the time needed to effect hemostasis and wound healing. For example, if the tines are made of a bioresorbable material, the material should not dissolve until at least after clotting, and in some embodiments, will not dissolve until the tissue has healed, at least partially.

The method of using the vascular closure device 10 is illustrated in the drawings. As mentioned above, the device 10 is used to seal an opening 30 in a blood vessel 40 or other body opening, such as might happen as a result of a surgical procedure. As shown in FIG. 1, the arterial sheath 20 may be first disposed at a puncture site 30 of a blood vessel 40 so that a distal end of the arterial sheath 20 is inserted into the interior of the blood vessel 40. The wire 100 with tines 70 and collar 80 may be positioned to be inserted into the proximal end 26 of the arterial sheath 20 for insertion into the blood vessel 40.

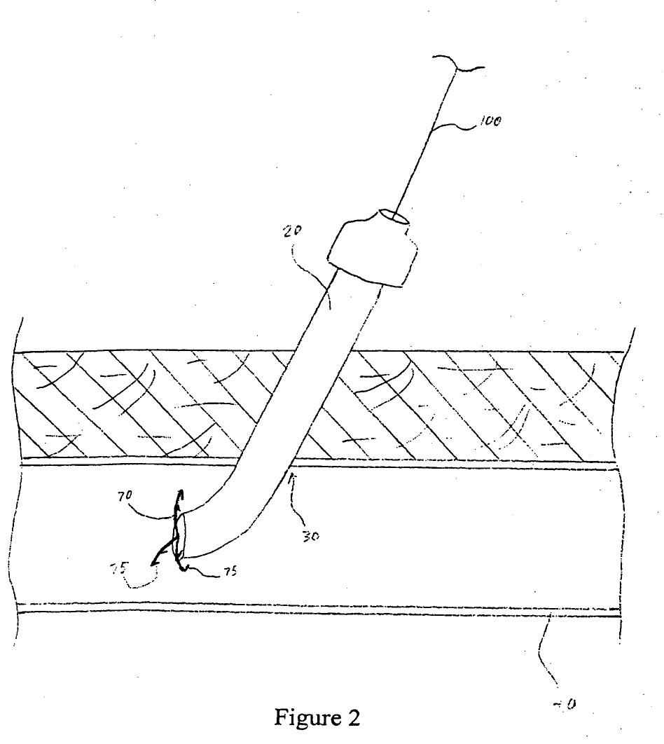

As shown in FIG. 2, the wire 100 may be advanced within the arterial sheath 20 placed at the puncture site 30 such that the tines 70 are advanced into the blood vessel 40 and allowed to expand into their open position within the blood vessel 40 from their closed position within the arterial sheath 20. In their open position, the tines 70 extend toward the walls of the blood vessel 40.

In one embodiment, the collar 80 can be situated over the base of the tines 70 during the insertion process through the sheath 20 such that the collar 80 keeps the tines from radially expanding into their open position. Once the tines 70 are delivered to the vessel 40, the collar 80 is moved longitudinally with respect to the tines 70 in the proximal direction, whereby the base of the tines 70 are no longer covered such that they may radially expand into their open position. In their open position, the tines 70 extend toward the sides of the puncture site 30. The tines 70 are then pulled against the interior of the puncture site 30 and the walls of the blood vessel 40. Additionally, the collar 80 may then move longitudinally with respect to the tines 70 in the distal direction, whereby the collar 80 slides back over the base of the tines 70, squeezing the tines 70 into their closed position as they retract.

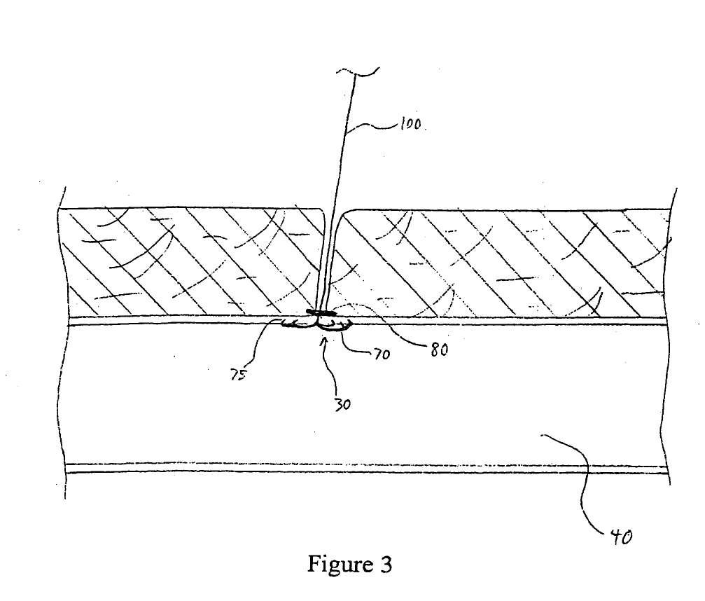

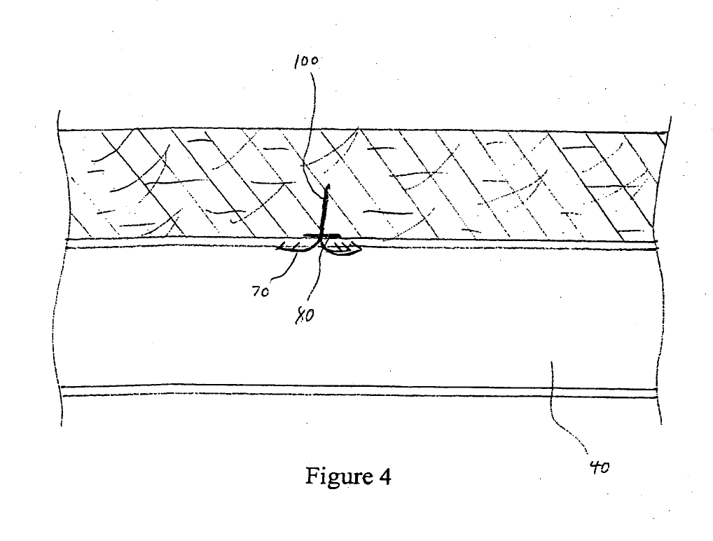

FIG. 3 shows that the arterial sheath 20 is removed, leaving the tines 70 remaining within the interior of the blood vessel 40. The tines 70 are on the distal side of the collar 80, which is situated just outside the blood vessel 40 in which the device 10 has been used to close the puncture site 30 in the blood vessel 40. As the tines 70 close toward each other, the barbs 75 grasp the surrounding tissue, appose it, and fold it together at the puncture site 30, thereby creating an obstruction of the flow of blood from the puncture site 30. The device 10 remains in place, with the tines 70 locked in a closed position by the collar 80. As shown in FIG. 4, the wire 100 may be cut so that the device may be left in place to hold the puncture site 30 closed. In another aspect, the device is then resorbed, by which time the puncture site 30 has sufficiently healed to avoid further bleeding. In another aspect, the collar 80 need not be resorbed.

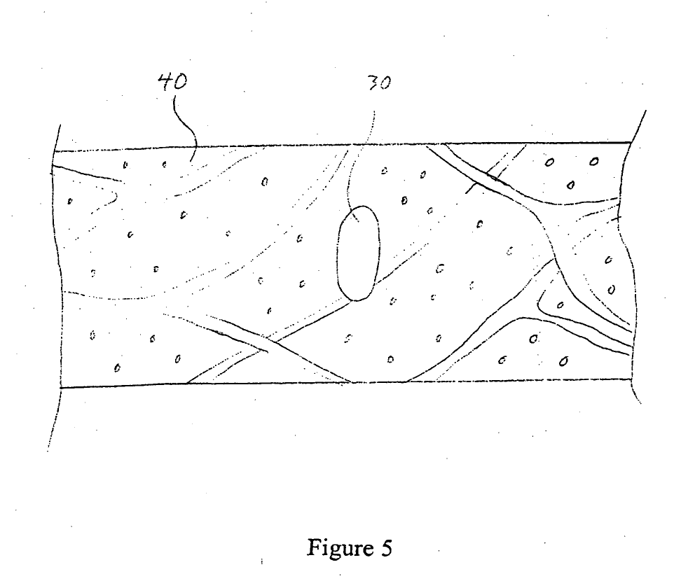

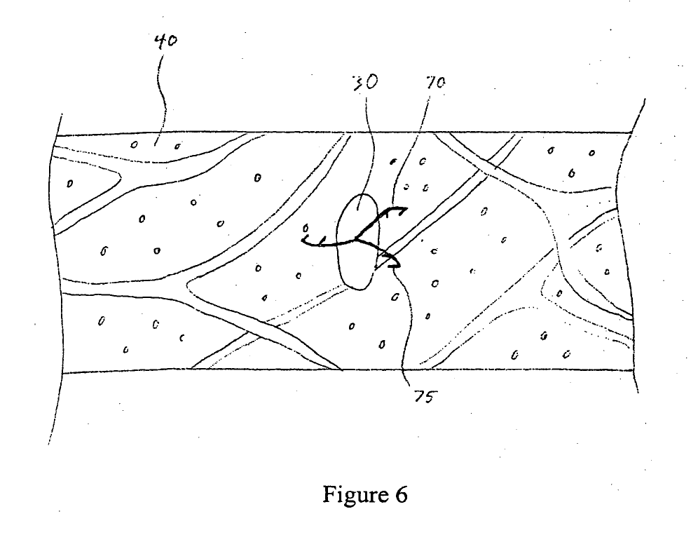

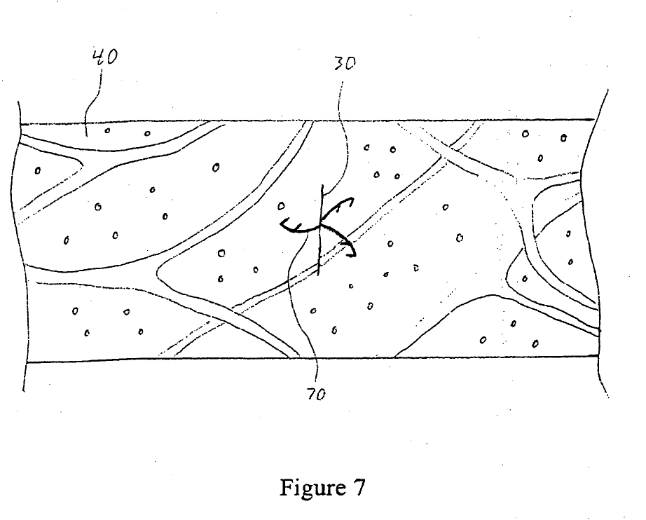

FIG. 5 shows a sectional view from within the blood vessel 40 with an open puncture site 30 before the device 10 has been inserted. FIG. 6 illustrates a sectional view from the interior of the blood vessel 40 after the tines 70 have been inserted through the opening of the puncture site 30 so that the barbs 75 on the tines 70 engage with the interior wall of the blood vessel 40. The tines 70 are then drawn together, such as by the collar 80, causing the puncture site 30 to be closed, as shown in FIG. 7.

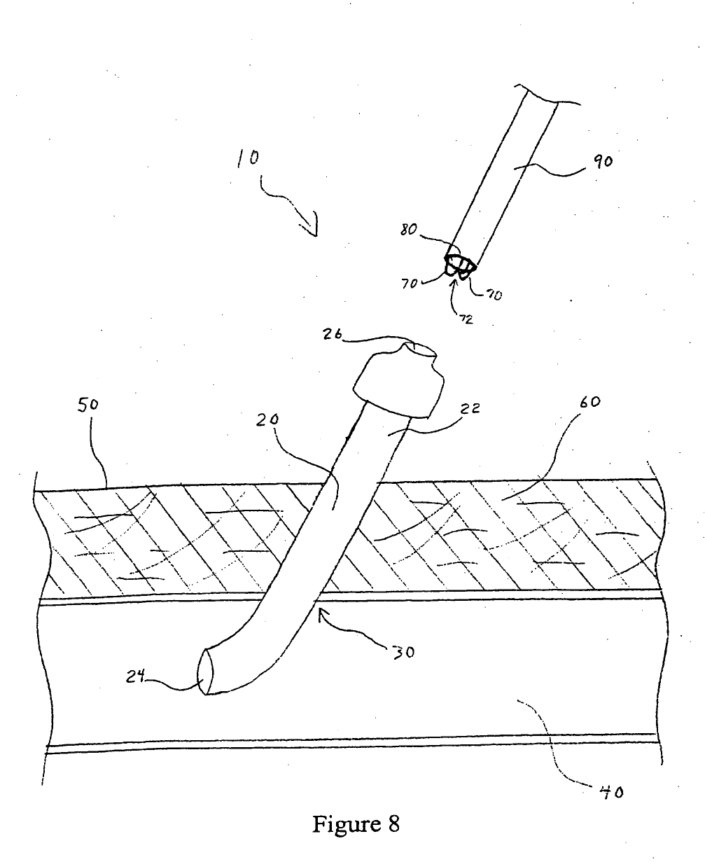

In another embodiment, the tines 70 are advanced into the vessel through a tube (such as the arterial sheath 20 or a catheter 90 in FIG. 8, for example), which is inserted into the blood vessel 40. Once advanced into the blood vessel, the tines 70 are allowed to expand into an open position inside the vessel 40. In this embodiment, the collar 80 does not keep the tines 70 in a closed position during insertion, but instead, the shape of the tube compresses the tines 70 for insertion. Once inserted, the tines 70 can be drawn back against the lumen interior. From this point, the tines can be retracted into a closed position using the collar thereby sealing the puncture site 30.

Referring to the drawings, it is apparent that the inwardly-directed barbs 75 of the tines 70 are adapted particularly for grabbing tissue at or closely adjacent to the interior wall surface of the blood vessel 40, and folding the tissue of the vessel 40 together inside the wall of the vessel 40.

Typically, the device may be left at the puncture site 30, because in some embodiments, the materials can be selected to be resorbed. The device can also be designed to be left in place permanently or removed after some period of time. In some embodiments, the collar and tines will be resorbed at different rates. For example, the collar may be resorbed earlier than the tines.

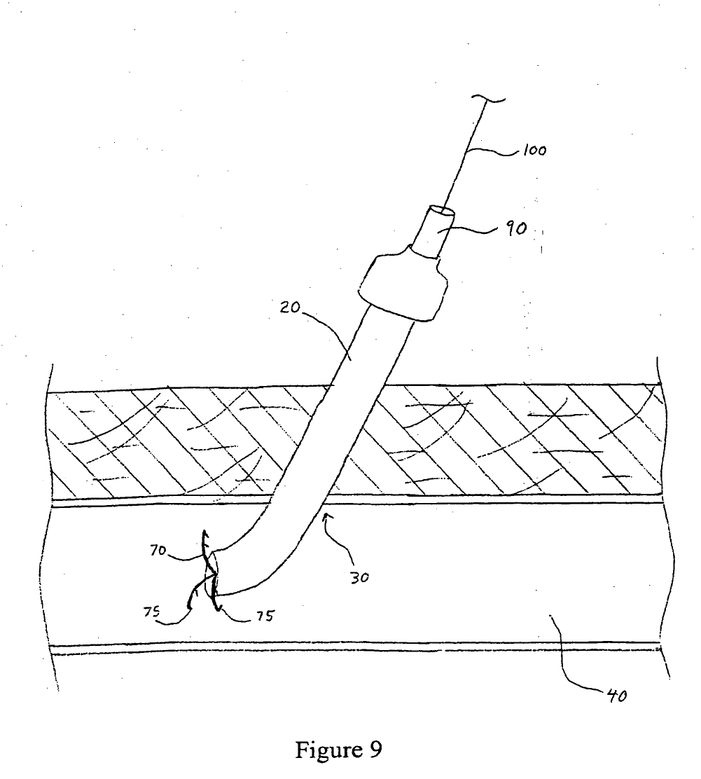

In another embodiment, the vascular closure device 10 comprises at least two, but preferably two pairs of, tines 70, a collar 80, and the catheter 90, as seen in FIG. 8. Use of the vascular closure device of the second embodiment commences after a surgical procedure that has created or exploited a puncture 30 in a blood vessel 40. As shown in FIG. 8, an arterial sheath 20 may be placed at the puncture site 30 and the catheter 90 may be sized such that it may be moved coaxially with respect to, and within, the arterial sheath 20. In this embodiment, the device 10 may include a catheter 90 to advance the suture or wire 100 and tines 70 through the proximal end 26 of the arterial sheath 20 so that the tines 70 may be advanced through the puncture site 30 and into the interior of the blood vessel 40, as shown in FIG. 9. The catheter 90 generally includes a body formed from a tube. The catheter 90 may also be employed to advance the collar 80 through the arterial sheath 20 and along the length of the suture or wire 100 toward the proximal end 72 of the tines 70.

A suture or wire 100 may be extended through the sheath 20 and attached to the device 10, and may be used to pull and guide the device through the sheath 20. In another embodiment, the suture may be used to guide the end of the catheter 90. By guiding the distal end 92 of the catheter, the device is advanced through the arterial sheath 20 in a collapsed state, being attached to the distal end of the catheter 90.

The catheter 90, the collar 80 within, and the tines 70 may be advanced through the sheath 20 and into position against the inner surface of the vessel wall 40. The tines 70 are on the distal side of the collar 80, which is situated just outside the blood vessel 40. The collar 80 may be situated over the base of the tines 70 such that the collar 80 keeps the tines 70 from radially expanding into their open position. The collar 80 may be sized such that it rests within the distal end of the catheter 90, coaxially movable in relation to the catheter 90. At this point, the distal end 92 of the catheter 90 and the collar 80 are disposed outside the opening of the puncture site 30, and the proximal end 94 of the catheter 90 extends away from the puncture site 30, toward, and possibly beyond, the skin 50, depending on its length.

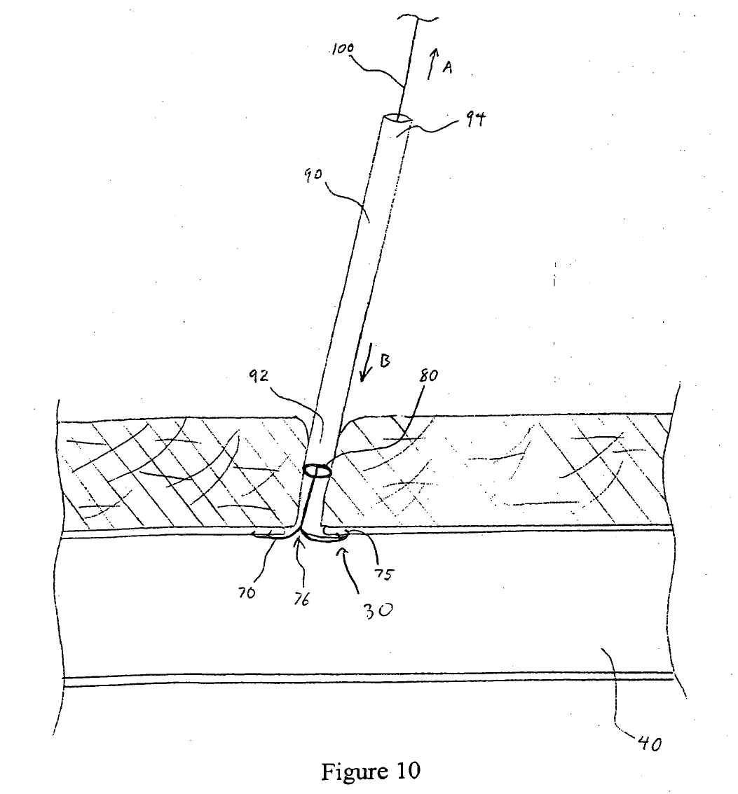

After the distal end 92 of the catheter 90 and the collar 80 have been placed at the opening of the puncture site 30, pressure is placed on the proximal end 94 of the catheter 90 such that the device is advanced past the distal end of the sheath 20 into the interior of the blood vessel 40, such that the tines 70 are no longer restrained and may radially expand outward into their open position. The device is then pulled back against the tip of the sheath 20, fully extending the tines 70. In their open position, the tines 70 extend toward the sides of the puncture site 30. The device and the sheath 20 are then retracted until the tines 70 make contact with the interior of the blood vessel wall 40. As shown in FIG. 10, the arterial sheath 20 is then removed and further tension on the suture 100 pulls the tines 70 firmly against the vessel wall.

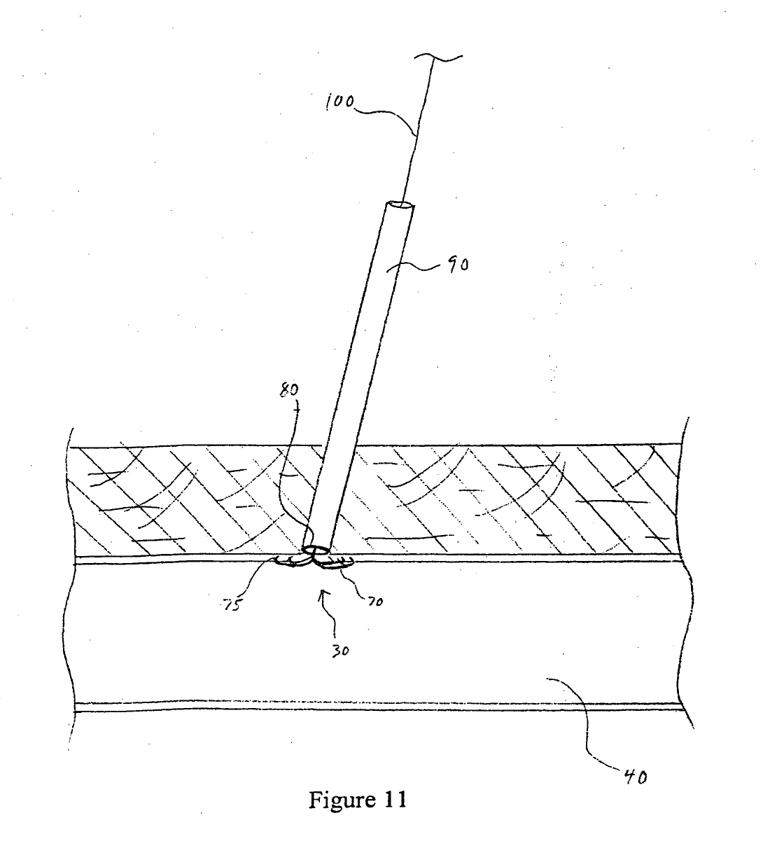

After the catheter 90 has been inserted into the arterial sheath 20 so that the tines 70 have been advanced through the puncture site 30, the arterial sheath may be removed, as shown in FIG. 10. Next, the puncture site 30 may be closed by drawing the tines 70 together. As the tines 70 close toward each other, the barbs 75 grasp the surrounding tissue, appose it, and fold it together at the puncture site 30, thereby creating an obstruction of the flow of blood from the puncture site 30. The collar 80 may be used to draw the tines 70 together. This may be accomplished by pulling the wire 100 in the direction indicated by arrow A in FIG. 10 so that the suture or wire 100 is pulled in a direction away from the proximal end 94 of the catheter 90. In addition, the collar 80 may be moved by advancing the catheter 90 in the direction indicated by arrow B in FIG. 10 so that the distal end 92 of the catheter 90 pushes the collar 80 towards the puncture site 30 and the base of the tines 70, as shown in FIG. 11. When the collar 80 slides over the base of the tines 70, the tines are locked together in place. Once the tines 70 have been used to close the puncture site 30 of the blood vessel 40, the catheter 90 may be removed and the suture or wire 100 may be cut so that the device is left to hold the puncture site 30 closed until the site is sufficiently healed.

The addition of the catheter 90 to the device 10 allows for additional maneuverability, control, and leverage over the placement and manipulation of the tines 70 and the collar 80. The device, however, does not require the catheter 90 for effective operation, and some practitioners may find that using their fingers alone for placement enables a more accurate placement.

The tines 70 and the collar 80 may act as physical obstructions to blood leakage, working in concert with the folded luminal walls. Hemostatic material may be placed on the collar 80 and/or the base of the tines 70 to aid in stemming blood flow through the puncture site 30 and to promote more effective and efficient hemostasis, until such point as the resorbable tines 70 and the collar 80 degrade following healing. Prior to such point of degradation, the anticoagulants on the collar 80 and the base of the tines 70 will have worn off, and the patient's innate coagulation mechanisms will have taken over the healing of the puncture site 30. In another embodiment, the collar 80 and/or the base of the tines 70 may be coated with a drug to promote healing of the puncture site and to prevent infection. For example, the collar 80 and/or the tines 70 may be coated with an antibiotic.

The present invention also provides methods of promoting hemostasis at a vascular opening and methods of closing an opening to a body cavity. These methods are described with reference to the operation of the closure device of the present invention. For example, in one embodiment, a method of closing an opening to a body cavity comprises: advancing at least two resilient tines through a body opening into a body lumen, wherein the at least two tines each have at least one barb adapted for catching on tissue and wherein the tines have an open position, wherein the at least two resilient tines are radially expanded and a closed position wherein the at least two resilient tines are radially contracted; retracting the tines in the open position against the interior tissue of the lumen, wherein the at least one barb catches on the tissue adjacent to the body opening; radially contracting the tines to the closed position wherein contracting pulls the edges of the tissue adjacent the opening together to close the body opening; and securing the tines in the closed position.

In another embodiment, the present invention provides a method for promoting hemostasis at a vascular opening comprising: providing percutaneous access to the tissue opening through an arterial sheath with an open distal end disposed within the vascular lumen and an open proximal end; providing at least two tines, wherein each tine is inwardly collapsible to be received in an arterial sheath and each tine has at least one barb for grasping vascular tissue, wherein the tines have an open position wherein the at least two resilient tines are radially expanded and a closed position wherein the at least two resilient tines are radially contracted into the sheath; advancing the tines through the sheath and into the vascular lumen so that the tines expand outwardly in the vascular lumen; retracting the tines so that the tines are pulled against the interior surface of the vascular lumen, wherein the tines catch on the tissue forming the interior surface of the vascular lumen; and advancing a collar to the exterior surface of the vascular opening, wherein the collar causes at least two resilient tines to radially contract in a manner to pull the edges of the vascular tissue together.

All the forgoing description and drawings have been limited to the use of the device in a blood vessel. However, the vascular closure device can also be used in many other applications including, but not limited to, closing intracardiac defects, repairing heart valves, and obliterating the left atrial appendage (LAA) to lessen the risk of stroke. Each application will be discussed, in turn, below.

As to intracardiac defects, the vascular closure device can be used to close atrial and ventricular septal defects. One example of an atrial septal defect is the patent foramen ovale (PFO), which is a defect in the wall, or septum, between the upper two chambers (atria) of the heart. During the development of a fetus, the atrial septum develops to eventually separate the left and right atria but a residual window between the atria (also known as the foramen ovale) remains open during fetal development to allow blood from the venous system to bypass the immature lungs and go to the systemic circulation system, because in the womb, the oxygenation of the blood is performed by the placenta (not the lungs). A layer of tissue begins to cover the foramen ovale during fetal development and the foramen ovale usually closes completely soon after birth. However, when the foramen ovale does not seal over, a PFO results, which under certain conditions may allow “right to left” shunting of blood across the atrial septum which may increase the risk of cryptogenic stroke and migraine headaches.

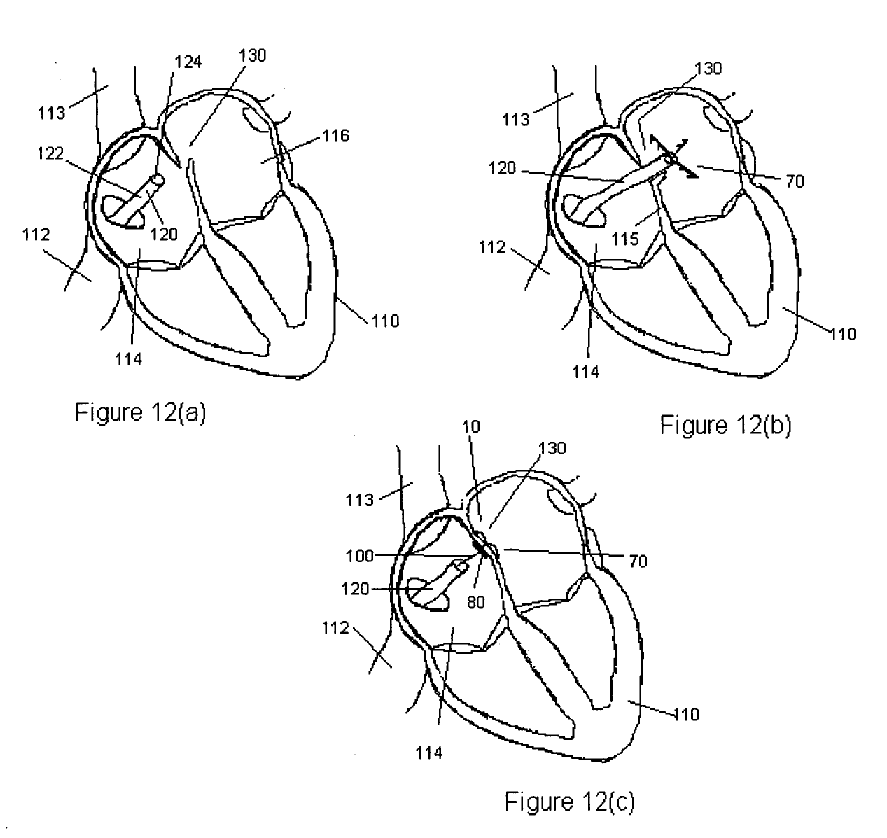

To close an intracardiac defect such as an atrial septal defect, the following procedure is used, as shown in FIGS. 12(a)-12(c). First, a catheter 120 is inserted into a large vein through a small incision made usually in the inner thigh and is advanced into the heart 110 though the inferior vena cava 112 into the right atrium 114 as seen in FIG. 12(a). Alternatively, a catheter 120 can be inserted into a vein in the neck area and advanced into the heart 110 through the superior vena cava 113. The catheter 120 may include a long, thin, flexible, and hollow tube 122, similar to catheter 90 in FIG. 8, which is used to guide the placement of the vascular closure device. The tube 122 has an open external or proximal end (not shown) that extends from a surgical entry site in the skin and an internal or distal end 124 which will extend into the heart. The vascular closure device is moved through the catheter to the heart and to the location of the septal defect 130.

As in other applications presented above, the vascular closure device 10 comprises at least two tines 70 attached to a suture or wire 100, and a collar 80. The catheter 120 is inserted in the inferior vena cava 112, pushes through the right atrium 114 and the septal defect 130 till the distal end 124 is located in the left atrium 116 of the heart 110 as shown in FIG. 12(b). The wire 100 with tines 70 and collar 80 may be positioned to be inserted into the proximal end of the catheter 120 for insertion into the heart 110 at the distal end 124.

As shown in FIG. 12(b), the wire 100 may be advanced within the catheter 120 such that the tines 70 are advanced into the left atrium 116 and allowed to expand to their open position within the left atrium 116 from their closed position within the catheter 120. In their open position, the tines 70 extend toward the walls of the septum 115 between the right atrium 114 and the left atrium 116. Next, the catheter 120 is retracted into the right atrium 114, leaving the tines 70 remaining within the interior of the left atrium 116. The collar 80 is located within the catheter 120 situated in the right atrium 114. In their open position, the tines 70 extend toward the sides of the septal defect 130. The tines 70 are then pulled against the edges of the septal defect 130 and the walls of the septum 115. This is accomplished by pulling the wire 100 toward the proximal end of the catheter 120 while catheter 120 pushes the collar 80 axially toward the tines 70 in the distal direction. As a result of this action, the collar 80 slides over the base of the tines 70, squeezing the tines 70 into their closed position as they retract.

Alternatively, the collar 80 can be situated over the base of the tines 70 during the insertion process through the catheter 120 such that the collar 80 keeps the tines from radially expanding into their open position. In this instance once the tines 70 are disposed in the left atrium 116, the collar 80 would be moved longitudinally with respect to the tines 70 in the proximal direction, whereby the base of the tines 70 are no longer covered such that they may radially expand into their open position. The tines 70 would then pulled against the interior of the septal defect 130 and the walls of the septum 115. This is accomplished by pulling the wire 100 toward the proximal end of the catheter 120 while catheter 120 pushes the collar 80 axially toward the tines 70 in the distal direction. As a result of this action, the collar 80 slides over the base of the tines 70, squeezing the tines 70 into their closed position as they retract.

FIG. 12(c) shows the heart 110 after the device 10 has initially been used to close the septal defect 130. As the tines 70 close toward each other, the barbs 75 grasp the surrounding tissue, appose it, and fold it together at the septal defect 130, thereby creating an obstruction of the flow of blood between the left and right atria. The device 10 remains in place, with the tines 70 locked in a closed position by the collar 80. The wire 100 may be cut by feeding any cutting mechanism known in the art, which can be fed through the catheter 120. Once the wire 100 is cut, the cutting mechanism can be retracted through the catheter 120 when the catheter is withdrawn. Alternatively, another catheter can be used to deliver the cutting mechanism to the site of the device 10 for cutting the wire 100. Consequently, the vascular closure device may be left in place to hold the septal defect 130 closed.

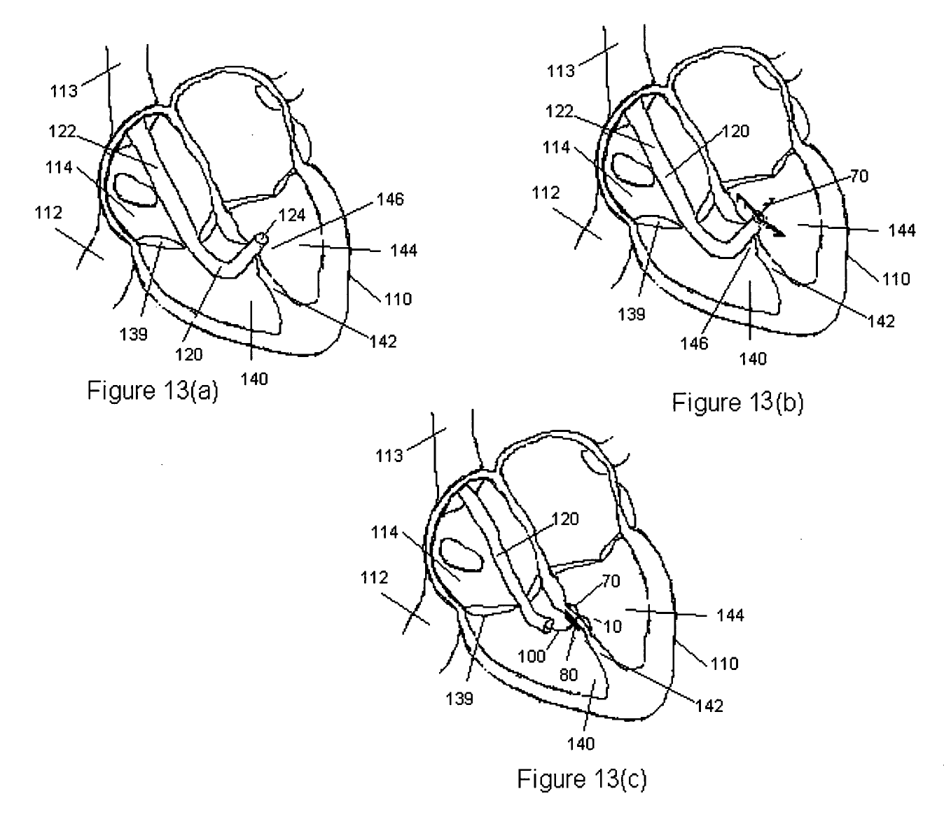

Beside PFO and other types of atrial septal defects, the vascular closure device can be used to close ventricular septal defects. In this case, there is an opening 146 in the septum 142 between the left ventricle 144 and the right ventricle 140 as seen FIGS. 13(a)-13(c). After the catheter 120 is inserted into a large vein and is advanced into the right atrium 114 of the heart 110 though either the inferior vena cava 112 or the superior vena cava 113, the catheter is pushed through the tricuspid valve 139, through the right ventricle 140, and through the septal defect 146 so that the distal end 124 of the catheter 120 is placed in the left ventricle 144. Then, the wire 100 with tines 70 and collar 80 may be positioned to be inserted into the proximal end of the catheter 120 for insertion into the heart 110.

As shown in FIG. 13(b), the wire 100 may be advanced within the catheter 120 placed at the left ventricle 144 near the septal defect 146 such that the tines 70 are advanced into the left ventricle 144 and allowed to expand to their open position within the left ventricle 144 from their closed position within the catheter 120. In their open position, the tines 70 extend toward the walls of the septum 142 between the right ventricle 140 and the left ventricle 144.

Next, the catheter 120 is retracted into the right ventricle 140, leaving the tines 70 remaining within the interior of the left ventricle 144. The collar 80 is located within the catheter 120 situated in the right ventricle 140. In their open position, the tines 70 extend toward the sides of the septal defect 146. The tines 70 are then pulled against the interior of the septal defect 146 and the walls of the septum 142. This is accomplished by pulling the wire 100 toward the proximal end of the catheter 120 while catheter 120 pushes the collar 80 axially toward the tines 70 in the distal direction. As a result of this action, the collar 80 slides over the base of the tines 70, squeezing the tines 70 into their closed position as they retract.

Alternatively, the collar 80 can be situated over the base of the tines 70 during the insertion process into the catheter 120 such that the collar 80 keeps the tines from radially expanding into their open position. In this instance once the tines have been disposed in the left ventricle 144, the collar 80 would be moved longitudinally with respect to the tines 70 in the proximal direction, whereby the base of the tines 70 are no longer covered such that they may radially expand into their open position. The tines 70 would then be pulled against the interior of the septal defect 146 and the walls of the septum 142. This is accomplished by pulling the wire 100 toward the proximal end of the catheter 120 while catheter 120 pushes the collar 80 axially toward the tines 70 in the distal direction. As a result of this action, the collar 80 slides over the base of the tines 70, squeezing the tines 70 into their closed position as they retract.

FIG. 13(c) shows the heart 110 after the device 10 has initially been used to close the septal defect 146. As the tines 70 close toward each other, the barbs 75 grasp the surrounding tissue, appose it, and fold it together at the septal defect 146, thereby creating an obstruction of the flow of blood between the left and right ventricles. The device 10 remains in place, with the tines 70 locked in a closed position by the collar 80. The wire 100 may be cut by feeding any cutting mechanism known in the art, which can be fed through the catheter 120. Once the wire 100 is cut, the cutting mechanism can be retracted through the catheter 120 when the catheter is withdrawn. Alternatively, another catheter can be used to deliver the cutting mechanism to the site of the device 10 for cutting the wire 100. Consequently, the vascular closure device may be left in place to hold the septal defect 146 closed.

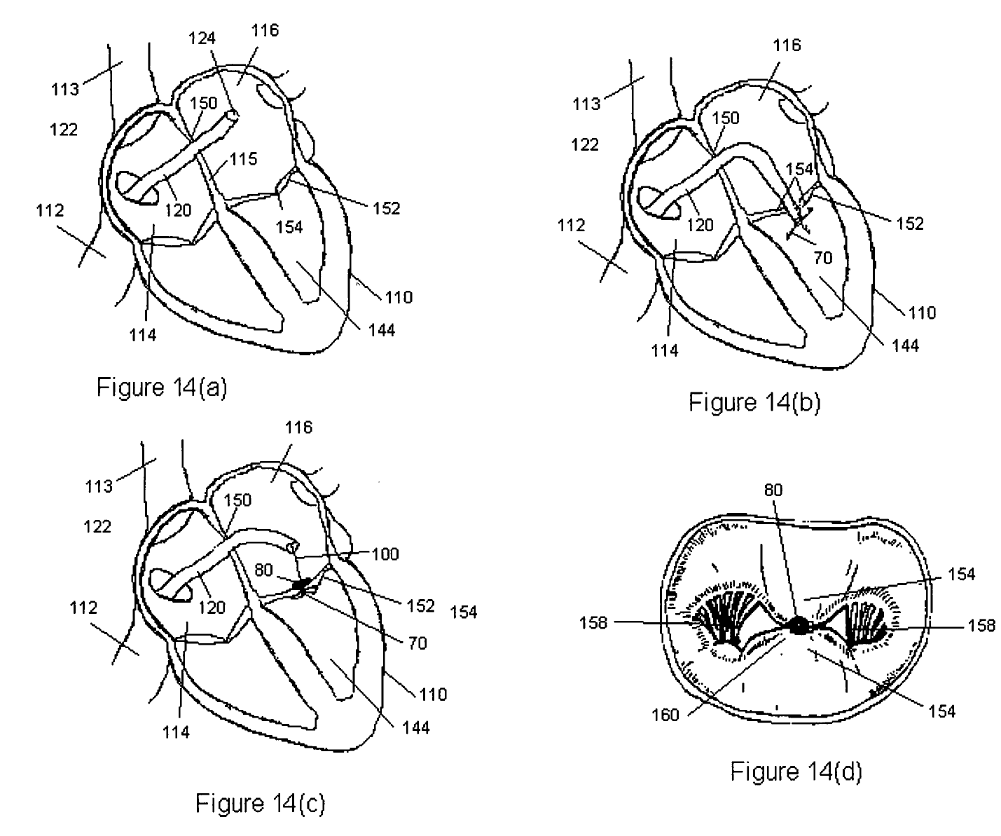

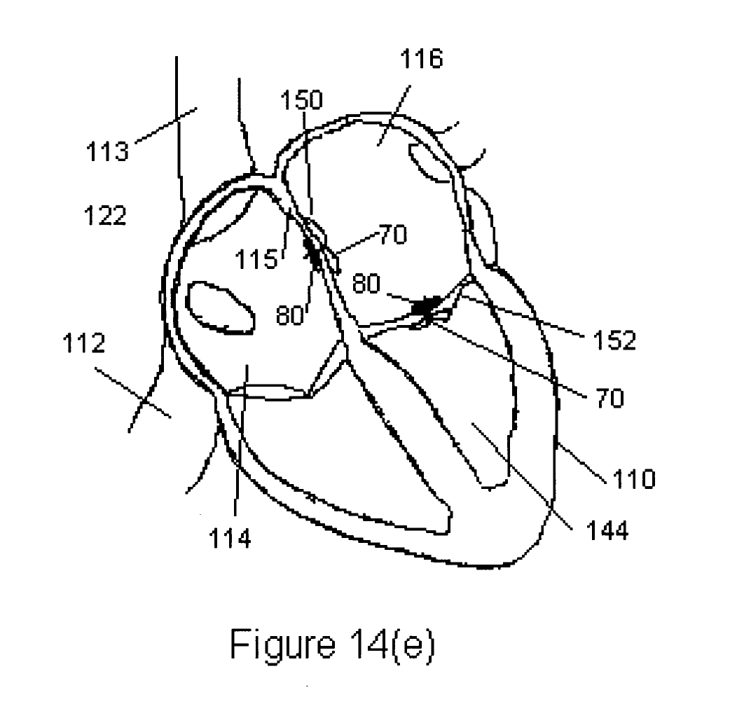

Another application that can utilize the vascular closure device is the repair of a heart valve for conditions such as mitral insufficiency (also known as mitral reguritation (MR)). For such a condition, the one way mitral valve does not seal completely and blood leaks back into the left atrium from the left ventricle. This leaking can cause the heart and lungs to swell. The vascular closure device can be used to mitigate the leaks by clamping the ends of the valve leaflets, and thus holding them together. For such a procedure, the catheter 120 is inserted and advanced into the heart 110 through the inferior vena cava 112 into the right atrium 114. Alternatively, the catheter 120 could be advanced into the heart 110 through the superior vena cava 113. At this point, a puncture 150 is then made through the fossa ovalis in the atrial septum 115, and the catheter 120 is advanced into the left atrium 116 as seen in FIG. 14(a). The catheter 120 is then pushed through the mitral valve 152 such that the distal end 124 is located in the left ventricle 144, as seen in FIG. 14(b). At which point, the wire 100 may be advanced within the catheter 120 such that the tines 70 are advanced into the left ventricle 144 and allowed to expand to their open position within the left ventricle 144 from their closed position within the catheter 120. In their open position, the tines 70 extend toward the center of the valve leaflets 154 of the mitral valve 152 between the left atrium 116 and the left ventricle 144.

In FIG. 14(c), the catheter 120 is retracted out of the left ventricle 144, leaving the tines 70 remaining within the interior of the left ventricle 144. The tines 70 are located near the center of the valve leaflets 154 of the mitral valve 152 on the distal side of the collar 80. The collar 80 can be located within the catheter 120 in the left atrium 116. In their open position, the tines 70 extend toward the sides of the center of the valve leaflets 154 of the mitral valve 152. The tines 70 are then pulled against the mitral valve 152. This is accomplished by pulling the wire 100 toward the proximal end of the catheter 120 while pushing the collar 80 longitudinally toward the tines 70 in the distal direction using the catheter 120, whereby the collar 80 slides over the base of the tines 70, squeezing the tines 70 into their closed position as they retract.

Alternatively, the collar 80 can be situated over the base of the tines 70 such that the collar 80 keeps the tines from radially expanding into their open position during the insertion process within the catheter 120. Once in the left ventricle 144, the collar 80 is moved longitudinally with respect to the tines 70 in the proximal direction, whereby the base of the tines 70 are no longer covered such that they may radially expand into their open position. Then, the catheter 120 and the collar 80 are placed in the left atrium 116. The tines 70 (in their open position) extend toward the sides of the center of the valve leaflets 154 of the mitral valve 152. The tines 70 are then pulled against the mitral valve 152. This is accomplished by pulling the wire 100 toward the proximal end of the catheter 120 while pushing the collar 80 longitudinally toward the tines 70 in the distal direction using the catheter 120, whereby the collar 80 slides over the base of the tines 70, squeezing the tines 70 into their closed position as they retract.

FIG. 14(c) shows the heart 110 after the device 10 has initially been used to partially close the mitral valve 152. As the tines 70 close toward each other, the barbs 75 grasp the surrounding tissue, appose it, and fold it together at the center of the mitral valve leaflets. The vascular closure device remains in place, with the tines 70 locked in a closed position by the collar 80. The wire 100 may be cut by feeding any cutting mechanism known in the art through the catheter 120. The wire 100 can be cut and the cutting mechanism can be retracted through the catheter 120 when the catheter is withdrawn. Alternatively a separate catheter can be used to deliver the cutting mechanism for cutting the wire 100. Consequently, the wire 100 may be cut so that the device may be left in place to hold the center 160 of the mitral valve 152 closed, as seen in FIG. 14(d).

The result of placing the vascular closure device at the center of the mitral valve 152 allows the valve to seal more effectively when it closes. Blood can still flow from the left atrium 116 to the left ventricle 144 when the valve is open because two openings 158 are formed on either side of the clamped center 160 as a result of the use of the vascular closure device, as seen in FIG. 14(d). It is noted that although one device has been used to repair the mitral valve 152 in FIGS. 14(a)-14(d), more than one device can be used on the valve 152, as necessary or desired. In addition, if it is necessary or desired, another vascular closure device comprising a collar 80, a wire 100, and tines 70 can be used to close the punture site 150 in the septum 115 which was used in the insertion process of the catheter 120. If such another set was used, there would be two vascular closure devices 10 in which one is placed at the center of the mitral valve 152 while another device is placed at the location of the punture site 150, as seen in FIG. 14(e).

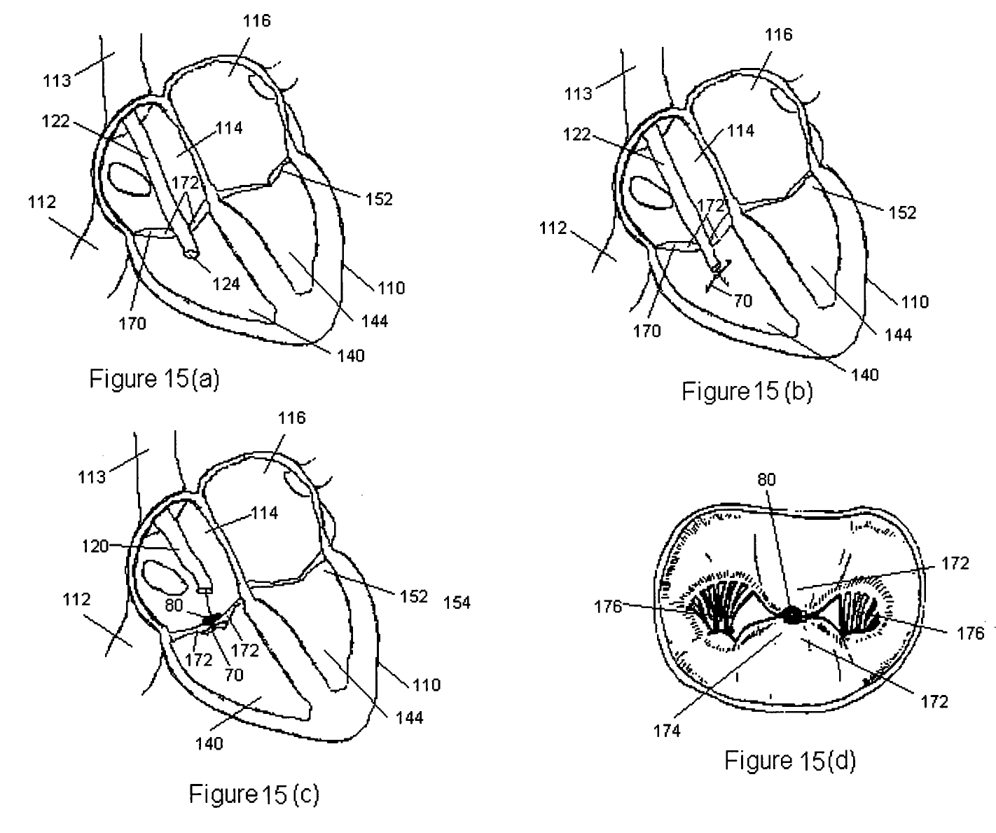

Similarly to the repair of the mitral valve, the tricuspid valve can also be repaired if there tricuspid regurgitation. The tricuspid valve is a valve the controls flow from the right atrium to the right ventricle. If the tricuspid valve is not completely sealing during its closure, the vascular closure device can be used to seal the valve leaflets in the same fashion as shown for the mitral valve. FIGS. 15(a)-15(d) show this application.

A catheter 120 is advanced into the heart 110 through the superior vena cava 113 into the right artium 114. Alternatively, the catheter 120 can be advanced into the heart 110 through the inferior vena cava 112. The catheter 120 is then pushed through the tricuspid valve 170 such that the distal end 124 is located in the right ventricle 140, as seen in FIG. 15(a). At which point, the wire 100 may be advanced within the catheter 120 such that the tines 70 are advanced into the right ventricle 140 and allowed to expand into their open position within the right ventricle 140 from their closed position within the catheter 120. In their open position, the tines 70 extend toward the center of the valve leaflets 172 of the tricuspid valve 170 between the right atrium 114 and the right ventricle 140, as seen in FIG. 15(b).

In the next step shown in FIG. 15(c), the catheter 120 is retracted out of the right ventricle 140, leaving the tines 70 remaining within the interior of the right ventricle 140. The tines 70 are located near the center of the valve leaflets 172 of the tricuspid valve 170 on the distal side of the collar 80, which is situated on the other side of the valve leaflets 172 of the tricuspid valve 170 in the right atrium 114. In their open position, the tines 70 extend toward the sides of the center of the valve leaflets 172 of the tricuspid valve 170. The tines 70 are then pulled against the tricuspid valve 170. This is accomplished by pulling the wire 100 toward the proximal end of the catheter 120 while moving the collar 80 longitudinally with respect to the tines 70 in the distal direction whereby the collar 80 slides over the base of the tines 70, squeezing the tines 70 into their closed position as they retract.

Alternatively, the collar 80 can situated over the base of the tines 70 such that the collar 80 keeps the tines from radially expanding into their open position during the insertion of the closure device into the catheter 120. Once the tines are inside the right ventricle 140, the collar 80 is moved longitudinally with respect to the tines 70 in the proximal direction, whereby the base of the tines 70 are no longer covered such that they may radially expand into their open position. In their open position, the tines 70 extend toward the sides of the center of the valve leaflets 172 of the tricuspid valve 170. The tines 70 are then pulled against the tricuspid valve 170. This is accomplished by pulling the wire 100 toward the proximal end of the catheter 120 while moving the collar 80 longitudinally with respect to the tines 70 in the distal direction whereby the collar 80 slides over the base of the tines 70, squeezing the tines 70 into their closed position as they retract.

FIG. 14(c) shows the heart 110 after the device 10 has initially been used to partially close the tricuspid valve 172. As the tines 70 close toward each other, the barbs 75 grasp the surrounding tissue, appose it, and fold it together at the center of the tricuspid valve leaflets. The device 10 remains in place, with the tines 70 locked in a closed position by the collar 80. The wire 100 may be cut by feeding any cutting mechanism known in the art, which can be fed through the catheter 120. Once the wire 100 is cut, the cutting mechanism can be retracted through the catheter 120 when the catheter is withdrawn. Alternatively, the cutting mechanism for cutting the wire 100 can be delivered by another catheter. Consequently, the wire 100 may be cut so that the device may be left in place to hold the center 174 of the tricuspid valve 170 closed as seen in FIG. 15(d).

The result of placing the vascular closure device at the center of the tricuspid valve 170 allows the valve to seal more effectively when it closes. Blood can still flow from the right atrium 114 to the right ventricle 140 when the valve is open because two openings 176 are formed on either side of the clamped center 174 as a result of the use of the vascular closure device, as shown in FIG. 15(d). It is noted that although one device has been used to repair the tricuspid valve 170 in FIGS. 15(a)-15(d), more than one device can be used on the valve 170, as necessary or desired.

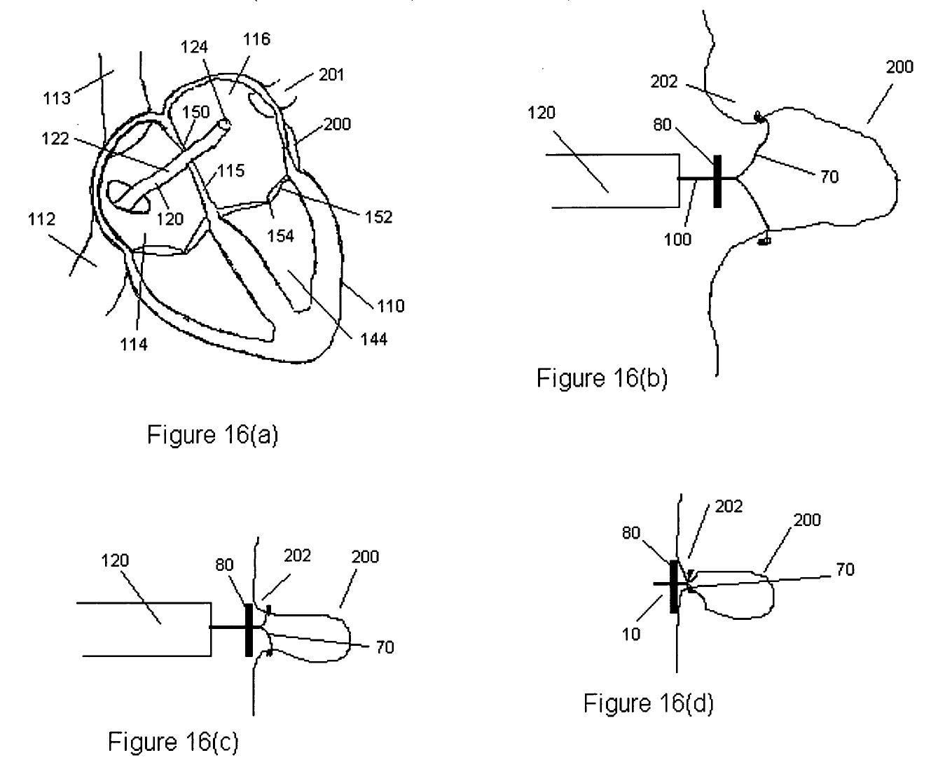

Another application that can be used with the vascular closure device 10 is the obliteration of the left atrium appendage (LAA). As seen in FIG. 16(a), the LAA is a cavity connected to a wall of the left atrium 116 between the mitral valve 152 and the root of the left pulmonary vein 201. The LAA normally contracts with the rest of the left atrium 116 during a normal cardiac cycle. However, when patients suffer from atrial fibrillation (an arrhythmia of the heart that results in a rapid and chaotic heartbeat), blood can remain stagnant inside the LAA. If this occurs, a thrombus can form in the stagnant blood, which can result in an eventual stroke if it leaves the LAA. Obliteration of the LAA can reduce the chances of stroke by closing the LAA so that there is an elimination or containment of the thrombus formed within the LAA. FIGS. 16(a)-16(d) disclose a method of using the vascular closure device in which the LAA can be closed.

Initially, a catheter 120 is inserted into a large vein and advanced into the heart 110 through the inferior vena cava 112 or the superior vena cava 113 into the right atrium 114. At this point, a puncture 150 is then made through the fossa ovalis in the atrial septum 115 and the catheter is advanced into the left atrium 116 whereby the catheter tube 122 is left in position as seen in FIG. 16(a). The catheter 120 is then pushed further into the left atrium 116 to the LAA 200. At which point, the wire 100 may be advanced within the catheter 120 such that the tines 70 are advanced into the LAA 200 and allowed to expand to their open position within the LAA 200 from their closed position within the catheter 120.

In the next step shown in FIG. 16(b), the catheter 120 is retracted out of the LAA 200, leaving the tines 70 remaining just within the interior of the LAA 200. The tines 70 and the collar 80 are located near the center of the opening of the LAA 200. The collar 80 is situated over the base of the tines 70 such that the collar 80 keeps the tines from radially expanding into their open position.

Next, the collar 80 is moved longitudinally with respect to the tines 70 in the distal direction, whereby the base of the tines 70 are no longer covered such that they may radially expand into their open position. In their open position, the tines 70 extend toward two or more points at the mouth 202 of the LAA 200. The tines 70 hook into the two or more points at the mouth 202 of the LAA. Subsequently, the collar 80 may then move longitudinally with respect to the tines 70 in the distal direction while pulling the wire 100 toward the proximal end of the catheter 120 whereby the collar 80 slides closer to the base of the tines 70, squeezing the tines 70 closer to their closed position as they continue retracting. As the tines 70 retract, the mouth 202 of the LAA 200 closes, as seen in FIG. 16(c).

The tines 70 and the collar 80 are configured such that they are capable of closing the mouth 202 of the LAA 200, as seen in FIG. 16(c). The collar 80 slides over the base of the tines 70, squeezing the tines 70 into their closed position, as seen in FIG. 16(d). The device remains in place, with the tines 70 locked in a closed position by the collar 80. The wire 100 may be cut by feeding any cutting mechanism known in the art, which is fed through the catheter 120. Once the wire 100 is cut, the cutting mechanism can be retracted through the catheter 120 when the catheter is withdrawn. Alternatively a different catheter can be used to deliver the cutting mechanism for cutting the wire 100 to the LAA site. Consequently, the wire 100 may be cut so that the device may be left in place to hold the mouth 202 of the LAA 200 closed.

Because the LAA 200 is closed by the vascular closure device 10, there is no opportunity for stagnant blood to collect. Thus, there is no thromus formed (or if there is one, it is trapped inside the LAA) and the chances of a stroke are reduced. It is noted that although one device has been used to close the LAA 200 in FIGS. 16(a)-16(c), more than one device can be used on the LAA 200, as necessary or desired. In addition, if it is necessary or desired, the another vasculare closure device comprising a collar 80, a wire 100, and tines 70 can be used to close the punture site 150 in the septum 115 that was used in the insertion process of the catheter 120 into the left artium 116. If such another set was used, there would be two vascular closure devices: one used to clamp the LAA 200 closed and one used to close the punture site 150.

The present disclosure shows the versatility of the vascular closure device. It can be used to close blood vessels and intracardiac defects, to repair heart valves, and to close the LAA. Given the disclosure of the present invention, one versed in the art would appreciate that there may be other embodiments and modifications within the scope and spirit of the invention that will suggest themselves to those skilled in the pertinent arts. For example, the number and configuration of the tines may be altered to suit differing surgical needs and their applications. Accordingly, all modifications attainable by one versed in the art from the present disclosure are to be included as further embodiments of the present invention, and should be considered within the spirit and scope of the present invention, as defined in the claims that follow.

What is claimed is:

1. A vascular closure device comprising:
(a) at least two tines including a distal end and a proximal end, wherein the at least two tines are joined at the proximal end, and wherein the at least two tines each have at least one barb adapted for catching tissue; and
(b) a collar with an opening receiving the proximal end of the at least two tines;
wherein the at least two tines expand radially outward to an open position when unrestricted, and wherein the collar can be moved from the proximal end of the at least two resilient tines towards the distal end of the at least two tines to radially contract the tines to a closed position.

2. The vascular closure device of claim 1, wherein the at least two tines are biodegradable.

3. The vascular closure device of claim 1, wherein the at least two tines are inwardly collapsible to be received into a vascular sheath.

4. The vascular closure device of claim 1, wherein the at least two tines are coated with a material to promote coagulation.

5. The vascular closure device of claim 1, wherein the at least two tines are coated with a hemostatic material.

6. The vascular closure device of claim 1, wherein the at least two tines are made from a hemostatic material.

7. The vascular closure device of claim 1, wherein the at least two tines are coated with a material to promote tissue growth.

8. The vascular closure device of claim 1, wherein the at least two tines are visible on an X-ray.

9. The vascular closure device of claim 1, wherein the at least two tines are made of memory-shaped alloy material.

10. The vascular closure device of claim 1, wherein when the at least two tines are in the open position, the at least two resilient tines each have at least two deflections wherein both deflections move the distal end of the tine away from the longitudinal axis of the tines and wherein the deflection at the distal end of the tines is greater than the deflection at the proximal end of the tines.

11. The vascular closure device of claim 10, wherein when the at least two tines are in the closed position, the most proximal deflection is substantially eliminated.

12. The vascular closure device of claim 1, wherein the collar is biodegradable.

13. The vascular closure device of claim 1, wherein the collar is coated with a material to promote coagulation.

14. The vascular closure device of claim 1, wherein the collar is coated with a material to promote tissue growth.

15. The vascular closure device of claim 1, wherein each tine has a plurality of barbs adapted for catching tissue.

16. A device for closing a vascular opening or other body cavity opening comprising:
(a) at least two tines including a distal end and a proximal end, wherein the at least two tines are joined at the proximal end, and wherein the at least two tines each have at least one barb adapted for catching tissue, and wherein the at least two tines are deflected outward from the longitudinal axis of the tines; and
(b) a collar with an opening receiving the proximal end of the at least two resilient tines;
wherein the collar can be moved from the proximal end of the at least two resilient tines towards the distal end of the two resilient tines to radially contract the tines thereby decreasing the deflection.

17. The vascular closure device of claim 16, wherein the at least two tines are biodegradable.

18. The vascular closure device of claim 16, wherein the at least two tines are inwardly collapsible to be received into a vascular sheath.

19. The vascular closure device of claim 16, wherein the at least two tines are coated with a material to promote coagulation.

20. The vascular closure device of claim 16, wherein the at least two tines are coated with a hemostatic material.

21. The vascular closure device of claim 16, wherein the at least two tines are made from a hemostatic material.

22. The vascular closure device of claim 1, wherein the at least two tines are coated with a material to promote tissue growth.

23. The vascular closure device of claim 16, wherein the at least two tines are visible on an X-ray.

24. The vascular closure device of claim 16, wherein the at least two tines are made of memory-shaped alloy material.

25. The vascular closure device of claim 16, wherein when the at least two tines are in the open position, the at least two resilient tines each have at least two deflections wherein both deflections move the distal end of the tine away from the longitudinal axis of the tines and wherein the deflection at the distal end of the tines is greater than the deflection at the proximal end of the tines.

26. The vascular closure device of claim 25, wherein when the at least two tines are in the closed position, the most proximal deflection is substantially eliminated.

27. The vascular closure device of claim 16, wherein the collar is biodegradable.

28. The vascular closure device of claim 16, wherein the collar is coated with a material to promote coagulation.

29. The vascular closure device of claim 16, wherein the collar is coated with a material to promote tissue growth.

30. The vascular closure device of claim 16, wherein each tine has a plurality of barbs adapted for catching tissue.

31. A vascular closure device comprising:
(a) a tube with an open distal end;
(b) a collar disposed on the distal end of the tube and including an opening for receiving at least two tines, wherein the collar is sized to be pushed by the tube when the tube is moved distally, and
(c) at least two tines, each of the tines inwardly collapsible to be received in an arterial sheath and including at least one barb adapted for grasping vascular tissue, wherein the tines are moveably connected to the opening of the collar
whereby, when unrestricted, the tines expand radially outward to an open position, and when the tube is moved distally, the collar is moved distally thereby moving the tines into the opening of the collar to radially contract the tines to a closed position.

32. The vascular closure device of claim 31, further comprising an arterial sheath including a proximal and distal end, wherein the tube is coaxially disposed in the arterial sheath and wherein the at least two tines are positioned at the distal end of the arterial sheath.

33. The vascular closure device of claim 31, wherein the collar comprises a bioresorbable material.

34. The vascular closure device of claim 31, wherein the collar comprises a hemostatic material.

35. The vascular closure device of claim 31, further comprising a cord connected to the at least two resilient tines.

36. The vascular closure device of claim 35, wherein the cord comprises a bioresorbable material.

37. The vascular closure device of claim 35, wherein the cord comprises a standard surgical suture.

38. The vascular closure device of claim 35, wherein the cord comprises a material selected from the group consisting of vicryl, polydioxalone, polypropylene, nylon, silk, and steel.

39. The vascular closure device of claim 35, wherein the tines are connected to the cable by a knot.

40. The vascular closure device of claim 35, wherein the tines are connected to the cable by an adhesive.

41. The vascular closure device of claim 31, wherein there are at least three tines.

42. The vascular closure device of claim 31, wherein the tines comprise a memory-shaped alloy.

43. The vascular closure device of claim 42, wherein the memory-shaped alloy comprises nitinol.

44. A method of closing an opening to a body cavity comprising:
(a) advancing at least two resilient tines through a body opening into a body lumen, wherein the at least two tines each have at least one barb adapted for catching on tissue and wherein the tines have an open position wherein the at least two resilient tines are radially expanded and a closed position wherein the at least two resilient tines are radially contracted;
(b) retracting the tines in the open position against the interior tissue of the lumen, wherein the at least one barb catches on the tissue adjacent to the body opening;
(c) radially contracting the tines to the closed position wherein contracting pulls the edges of the tissue adjacent opening together to close the body opening; and
(d) securing the tines in the closed position.

45. The method of claim 44, wherein the securing is performed using a collar adapted to receive the at least two resilient tines.

46. The method of claim 45, wherein the collar is biodegradable.

47. The method of claim 45, wherein the collar comprises a material that promotes coagulation.

48. The method of claim 45, wherein the collar is coated with a drug.

49. The method of claim 48, wherein the drug is an antibiotic.

50. The method of claim 44, wherein the securing is performed using at least one suture.

51. The method of claim 44, wherein the at least two tines are inwardly collapsible for passing through a tube.

52. The method of claim 44, wherein the at least two tines are biodegradable.

53. The method of claim 44, wherein the at least two tines are coated with a substance to promote coagulation.

54. The method of claim 44, wherein the at least two tines are coated with a substance to promote tissue growth.

55. The method of claim 44, wherein the at least two tines are coated with a drug.

56. The method of claim 55, wherein the drug is an antibiotic.

57. The method of claim 44, wherein the body opening is an atrial or ventricular septal defect in a heart and the body lumen is a chamber of the heart.

58. The method of claim 44, wherein the body opening is the mouth of a left atrium appendage in a heart and the body lumen is the left atrium appendage.

59. The method of claim 44, wherein the body opening is a heart valve and the body lumen is a chamber of the heart.

60. A method for promoting hemostasis at a vascular opening comprising:
(a) providing percutaneous access to the tissue opening through an arterial sheath including an open distal end disposed within the vascular lumen and an open proximal end;
(b) providing at least two tines, wherein each tine is inwardly collapsible to be received in an arterial sheath and each tine has at least one barb for grasping vascular tissue, wherein the tines have an open position wherein the at least two resilient tines are radially expanded and a closed position wherein the at least two resilient tines are radially contracted; into the sheath;
(c) advancing the tines through the sheath and into the vascular lumen so that the tines expand outwardly in the vascular lumen;
(d) retracting the tines so that the tines are pulled against the interior surface of the vascular lumen, wherein the tines catch on the tissue forming the interior surface of the vascular lumen; and
(e) advancing a collar to the exterior surface of the vascular opening, wherein the collar causes at least two resilient tines to radially contract in a manner to pull the edges of the vascular tissue together.

61. The method of claim 60, further comprising withdrawing the arterial sheath.

62. The method of claim 61, wherein the withdrawing occurs after (c).

63. The method of claim 60, wherein the collar is advanced using a tube with the collar disposed on the distal end of the tube.

64. The method of claim 60, wherein a cord is attached to the tines to retract the tines.
